(19) United States
(12) Patent Application Publication
(10) Pub. No.: US 2024/0242848 A1
(43) Pub. Date: Jul. 18, 2024

(54) Group Outcome Results Analysis Using Automatically Generated Control Group for Outcome Comparison

(71) Applicant: Aon Consulting, Inc., Chicago, IL (US)

(72) Inventors: Todor Penev, Atlanta, GA (US); Robert Tate, Atlanta, GA (US); Yunjie Zhao, Portland, OR (US)

(73) Assignee: Aon Consulting, Inc., Chicago, IL (US)

(21) Appl. No.: 18/410,630

(22) Filed: Jan. 11, 2024

Related U.S. Application Data
(60) Provisional application No. 63/438,675, filed on Jan. 12, 2023.

(51) Int. Cl. G16H 50/70

(52) U.S. Cl. CPC G16H 50/70

(57) ABSTRACT

In an illustrative embodiment, systems and methods for automatically building a control population closely matched to a member population and applying the control population in establishing medical outcome comparison metrics with the member population includes accessing demographic information for the member population, classifying the members into demographic groups of demographic categories including age, gender, and/or geography, accessing medical data of the member population, and classifying the members into condition groups of medical condition categories. The systems and methods may include using the demographic and medical condition classifications to match members of the member population to individuals of a control population. The systems and methods may include analyzing the member population in view of the matching individuals of the control population in relation to health outcomes, medical efforts, and/or healthcare utilization.

### RELATED APPLICATIONS

The present application claims priority to U.S. Provisional Patent Application No. 63/438,675 filed Jan. 12, 2023. The above identified application is hereby incorporated by reference in its entirety.

### BACKGROUND

Benchmarking performance of health initiatives, including innovative features of group medical plans, faces a number of difficulties. Because of inconsistency of variables such as, in some examples, developed interventions, changes in health circumstances of a covered group of individuals, or movements in the population of the covered group of individuals, comparisons made between prior years and a present year do not necessarily identify benefits or detriments due to change in the health initiatives alone. Prior solutions have involved gross oversimplification of the variables through the application of assumptions. The inventors identified a need for providing an improved system that allows for benchmarking groups of participants in a variety of health initiatives, such as particular medical plans, lifestyle programs, care management programs, and/or wellbeing initiatives, to a control or comparison group.

### SUMMARY OF ILLUSTRATIVE EMBODIMENTS

The inventors produced systems and methods for providing control group generation to be used in outcome comparison for analyzing and predicting the outcome of a medical treatment based on information relating to the covered group of individuals having a different medical plan than the topic medical plan in review. The methods and systems have greater application beyond medical group and treatment plan analysis and considerations such as e.g., medical condition care outcome prediction.

Aspects of the present disclosure are designed to provide information about and predict the relative effectiveness of healthcare services used by people. Information about their healthcare effectiveness and utilization may be generated by methods and systems described herein. The methods and systems may then continue to find other people of similar demographics (e.g., age, gender, and location, for example), who have similar health problems. We refer to these other people as the “comparison group” or as the “control group”—these terms can be used interchangeably.

Aspects of the present disclosure are designed to provide information about the relative effectiveness of healthcare initiatives on people depending on their health management, e.g., depending on whether or not they participate in wellbeing, wellness, disease management, and/or other health management programs. Participants in those programs, for example, may be matched to similar non-participants, using methods and systems described herein. Their healthcare outcomes and utilization of hospital, outpatient, and pharmaceutical services may be compared to discover whether and to what extent program participation contributes to improving the health of the respective people, thereby reducing healthcare efforts and/or utilization. Thus, the present disclosure provides a technical approach for tracking the results of health management efforts and related health improvements.

In some embodiments, the analysis performed according to the invention allows to employ the results for further addressing audit and benefit strategy questions, such as:
Are the vendors hired to help manage healthcare initiatives and utilization accurately reporting the impact of their services on healthcare outcomes and utilization?
Does a given annual healthcare insurance plan strategy focus on the highest-potential areas for improvement in the plan?
How do healthcare outcomes and utilization vary between members of different age groups, gender, and geographic locations?
How do healthcare outcomes and utilization vary according to whether members have one or more chronic conditions of interest, such as (but not limited to) musculoskeletal problems, coronary heart disease, diabetes, asthma, and other conditions?

Thus, the innovations described in the present disclosure provide an improved system for tracking health care outcomes and health care impacts, thus improving offerings, initiatives, and interventions to best manage and improve the health of members while at the same time keeping the costs at a relatively low level.

In one aspect, systems and methods of the present disclosure involve accessing medical data, medical plan data, and demographics data of a large population of individuals and pairwise matching each member of a covered group of individuals to a similar member of the large population of individuals. The size of the population, in some examples, may be thousands of individuals, tens of thousands of individuals, hundreds of thousands of individuals, at least one million individuals, at least ten million individuals, or over twenty million individuals. The matching may be applied across demographic, socioeconomic, and medical condition variables to identify one or more closest matches to each member. For example, a closest one, two, three, four, five, or more (e.g., as many as ten individuals) may be identified per covered member as being closest matching members.

In some embodiments, pairwise matching of each member to one or more closest matching members includes applying dimensionality reduction to cluster demographic and medical condition information. For example, demographic information may be broadened to a metropolitan area, an age range, and/or a household income range. In another example, certain medical conditions may be clustered, such as certain skin rash conditions, certain heart conditions, etc. In grouping demographic and/or medical condition variables, for example, the potential matching variables may be dimensionally reduced. Dimensionality reduction of variables, in some examples, may result in from 100 to 500 variables, or about 200 variables.

In some embodiments, machine learning is used for dimensionality reduction. For example, machine learning algorithms may identify a closest N individuals to a member. If an individual is not identified using the tightest grouping of one or more of the demographic, socioeconomic, and/or medical condition variables, the machine learning analysis may expand one or more groupings to identify one or more matching individuals. For example, if a matching individual is not identified within a city geographic region, the geographic region may be expanded to a county region.

In some embodiments, machine learning is used to identify the most relevant factors to analysis, thereby aiding in dimensionality reduction. In identifying the most relevant factors, for example, the present disclosure describes an improved system for identifying and quantifying actionable health care impacts, thus providing an avenue to direct future medical case studies related to various healthcare initiatives and interventions. In some examples, the most relevant factors may include and/or provide insights related to most common side effects to a new medical intervention, most common injuries caused by a particular medical procedure, and/or diseases/disorders unusually prevalent to a particular demographic (e.g., population, geographic region, etc.). For example, machine learning may be used to identify at least about 10 to 100 medical condition variables that create the greatest need for intervention among the population.

In some embodiments, to perform dimensionality reduction, machine learning is used to group medical coding information into classifications related to particular diseases and/or disorders. Many medical coding taxonomies include many similar codes that apply to a particular overarching disease/disorder (e.g., HIV infection) or set of diseases/disorders (e.g., heart disease). Thus, a technical difficulty exists in the field related to analyzing medically coded data due to the specificity at which medical procedures, interventions, and pharmaceuticals are coded. To see the forest for the trees, the inventors created a solution for applying machine learning to reduce the tens of thousands of codes to manageable and actionable classifications. The classifications, further, may include sub-divisions (e.g., sub-divisions of heart disease, sub-divisions of cancer, etc.), thereby providing varying gradations of analysis specificity to the methods and systems described herein.

In identifying the most relevant factors, for example, the present disclosure describes an improved system for identifying and quantifying actionable health care impacts, thus providing an avenue to direct future medical case studies related to various healthcare initiatives and interventions. In some examples, the most relevant factors may include and/or provide insights related to most common side effects to a new medical intervention, most common injuries caused by a particular medical procedure, and/or diseases/disorders unusually prevalent to a particular demographic (e.g., population, geographic region, etc.). For example, machine learning may be used to identify at least about 10 to 100 medical condition variables that create the greatest need for intervention among the population.

In some embodiments, benchmark metrics are derived from both the member group population and the matching individuals (control) population. The metrics can include, in some examples, outcomes (e.g., costs, disease rates, mortality rates, etc.) across each population, perception ratings (e.g., indication of satisfaction) across each population, medical outcomes across each population, healthy behavior changes across each population, prescription medication use across each population, hospitalization across each population, mortality rates across each population, and/or correlates of work productivity, such as days missed from work or lower self-reported or otherwise-evidenced performance of job duties while at work. Individual areas of metrics may be broken down into a variety of analyses, such as cost per gender, cost per age bracket, and/or cost in budget area (e.g., inpatient, outpatient, pharmaceutical, etc.). The metrics, for example, can demonstrate measurable effects of health programs, new medical interventions, fitness programs, and/or lifestyle changes on medical system usage and associated healthcare outcomes.

The benchmark metrics, in some embodiments, may be analyzed by the system. The system may then provide this analysis as part of a report format indicating differences between the member population and the control population. The report may summarize the analysis using graphics and/or numerical summaries comparing the member population to the control population across the various benchmark metrics.

The forgoing general description of the illustrative implementations and the following detailed description thereof are merely exemplary aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a block diagram of an example environment and platform for performing health care efficiency analyses using an automatically identified control population:

FIG. 2 is a flow chart of an example method for automatically matching a target group population with a control group population:

FIGS. 3A and 3B are classification diagrams illustrating example classification groupings for matching a target group population with a control group population:

FIG. 4 is an operational flow diagram of an example process for automatically generating a report presenting comparison metrics between outcomes of members of a target group and outcomes of members of an automatically generated control group:

FIGS. 5A through 5C illustrate example bar graph outputs of comparison metrics between a target group and an automatically generated control group:

FIG. 6 illustrates example table outputs of comparison metrics between a target group and an automatically generated control group:

FIG. 7 illustrates an example comparison table representing difference in prevalence in chronic conditions between a target group and an automatically generated control group:

FIGS. 8A through 8C illustrate example graphic outputs of comparison metrics over time between a target group and an automatically generated control group: and

FIGS. 9A through 9C illustrate a flow chart of an example method and sub-methods for identifying features for matching members of a target population with members of a control population and for performing the matching.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

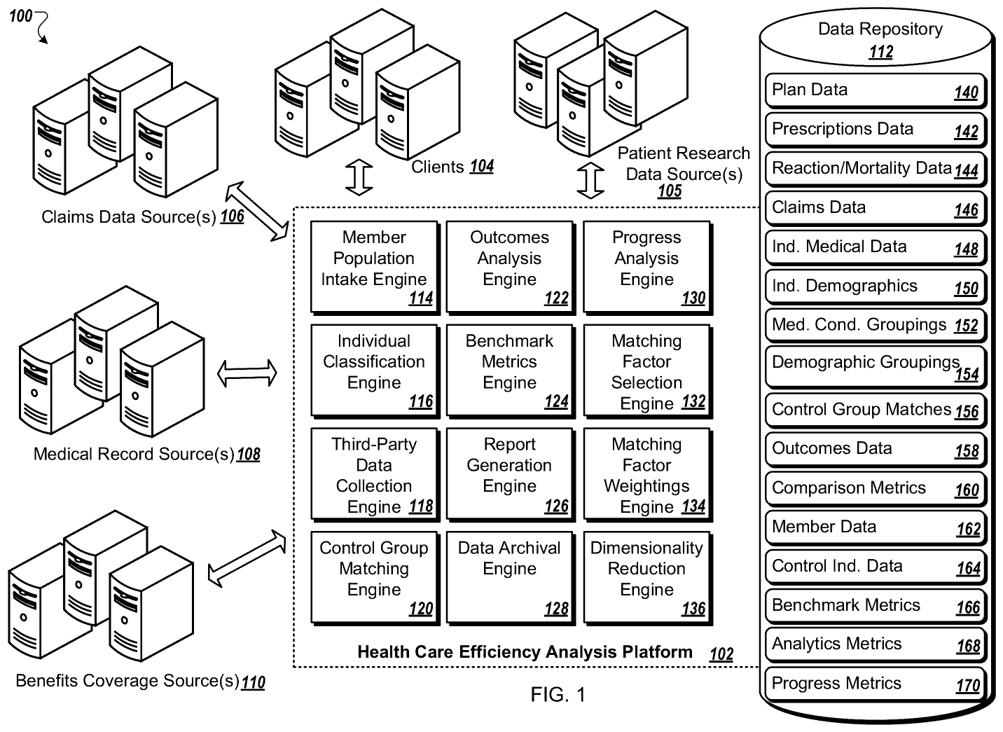

FIG. 1 is a block diagram of an environment 100 and health care efficiency analysis platform 102 for performing health care efficiency analyses using an automatically identified control population. At the core of the platform 102 is a control group matching engine 120 used to automatically match members of a target population with members of a control population based on both demographic characteristics and medical characteristics. The control group matching engine 120 may match on tens or hundreds of characteristics, in part through applying ranges, groupings, and other combinations to the characteristics for dimensionality reduction. Further, the control group matching engine 120 may match multiple control individuals to each target individual to reduce outlier results and/or to increase the odds of data availability regarding medical outcomes for at least one of the N control individuals spanning a number of years. In this manner, for example, the platform 102 may provide outcome efficiency feedback over time. The platform 102 may be used by clients 104 to compare outcomes of the target population to a state-of-the art or industry standard represented by the automatically generated control group of individuals. The clients 104, in one example, may be benefits plan providers submitting a target group of members of a benefits plan for comparison to a benefits plan industry standard represented by the automatically generated control group of individuals. In another example, the clients 104 may be medical device manufacturers, pharmaceutical companies, or other corporations conducting efficacy studies by comparing a target group of patients to a control group of patients having similar demographic characteristics and medical concerns, including the key concern of the efficacy study, but lacking the intervention provided by the client 104.

To enable comparisons, in some implementations, the health care efficiency analysis platform 102 obtains plan data 140 from one or more benefits coverage sources 110, prescriptions data 142 and/or individual medical data 148 from one or more medical record sources 108, and/or claims data 146 from one or more claims data sources 106. The plan data 140, for example, may identify sources 110 for the claims data 146 in the circumstance where members of the target group receive reimbursement through multiple sources. Further, the plan data 140, in some embodiments, may be used in matching target group patients to control group patients. For example, the target group may have a certain level of coverage (e.g., actuarial value of plan offering) which is matched to a range of similar coverages provided to the control group members.

Using the collected records, in some implementations, the health care efficiency analysis platform 102 derives outcome data such as outcome data 158 to use in comparison metrics 160 between the target group and the control group. The outcome data may be plan-neutral such that provider charges, rather than insurance payments, are considered in deriving cost-based outcome data 158. In the event of a significant difference between target group plan coverage and plan coverage provided to certain members of the control group, in some embodiments, the outcome data 158 may be adjusted for this discrepancy in coverage levels.

In some implementations, a member population intake engine 114 obtains member data 162 regarding a number of members of the target group from one of the clients 104. The member population intake engine 114 may confirm completeness of member information (e.g., demographics and/or medical information). In another example, the member population intake engine 114 may link at least a portion of demographics information and/or medical information from external sources, such as the claims data source(s) 106 and/or the medical record source(s) 108. For example, the member population intake engine 114 may query one or more external sources to retrieve additional information regarding one or more members of the target group. In another example, a third-party data collection engine 118 may access third party sources 106, 108 to obtain additional information regarding members of the target group. The member population intake engine 114 may store member information as member data 162 in a data repository 112, such as a database.

In some implementations, the third-party data collection engine 118 collects information from external data sources related to general health and well-being statistics, for example for grouping members of a population based on socio-economic factor(s), chronic condition factor(s), and/or environmental exposure factor(s). In a first illustration, the third-party data collection engine 118 may collect socio-economic factor data such as Area Deprivation Index (ADI) data to determine socio-economic (e.g., ADI) categorizations for regions in which members of a population live and/or work. The University of Wisconsin developed the ADI to compare census block groups (e.g., neighborhoods) by relative socioeconomic disadvantage on a scale from 1 to 100. The socio-economic factor data may be aggregated by the platform 102, in some embodiments, into courser groupings, such as, in an illustrative example, a high ADI risk (e.g., greater than 67), medium ADI risk (e.g., between 34 and 66) and low ADI risk (e.g., less than or equal to 33). Individuals living in high ADI risk areas tend to experience higher utilization of emergency room services and lower utilization of preventative services. The high ADI risk areas are associated with increased risk for a number of chronic conditions (e.g., diabetes, hypertension, asthma) as well as higher tobacco consumption. In a second illustration, the third-party data collection engine 118 may collect Clinical Classifications Software Refined (CCSR) diagnoses aggregations by the Agency for Healthcare Research and Quality (AHRQ) of Rockville, MD. The CCSR diagnosis groupings, for example, may be used to group members by clinical condition (e.g., chronic condition) groupings based on diagnosis codes found, in some examples, in the claims data 146 and/or the individual medical data 148. In a third illustration, environmental exposure data may be collected by the third-party data collection engine 118 to quantify environment exposure factors such as, in some examples, water quality data, air quality data, radon exposure data, etc.

The third-party data collection engine 118, in some implementations, collects information from external data sources related to member participation (or lack thereof) in one or more vendor programs. The vendor programs, in some examples, may include healthy eating programs, new medical interventions, fitness programs, and/or lifestyle coaching programs.

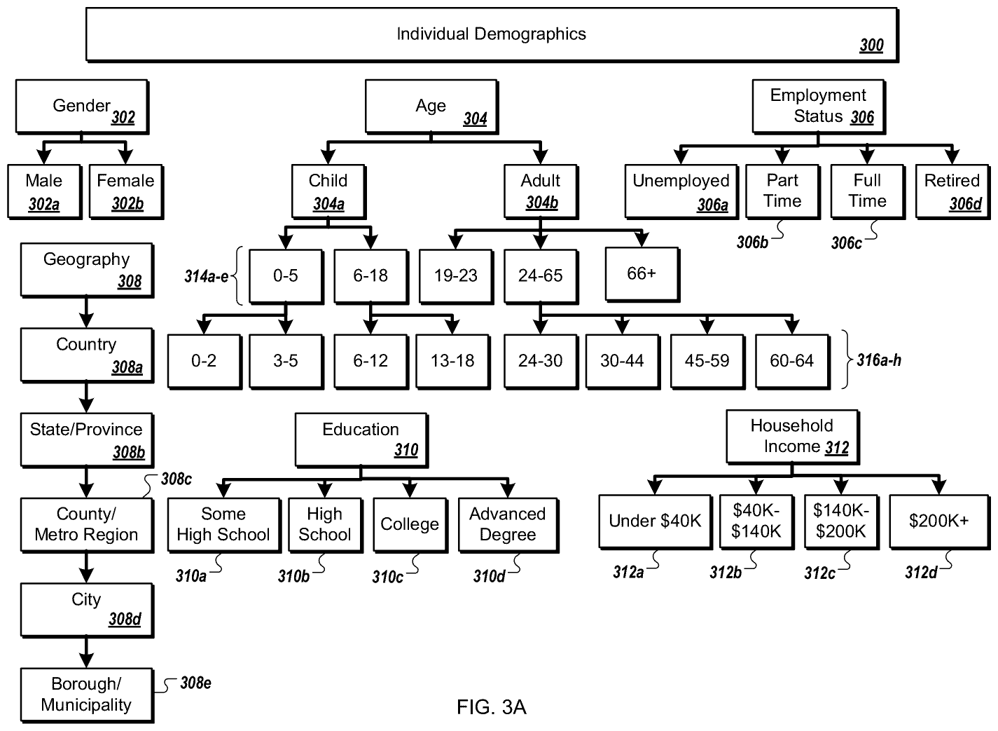

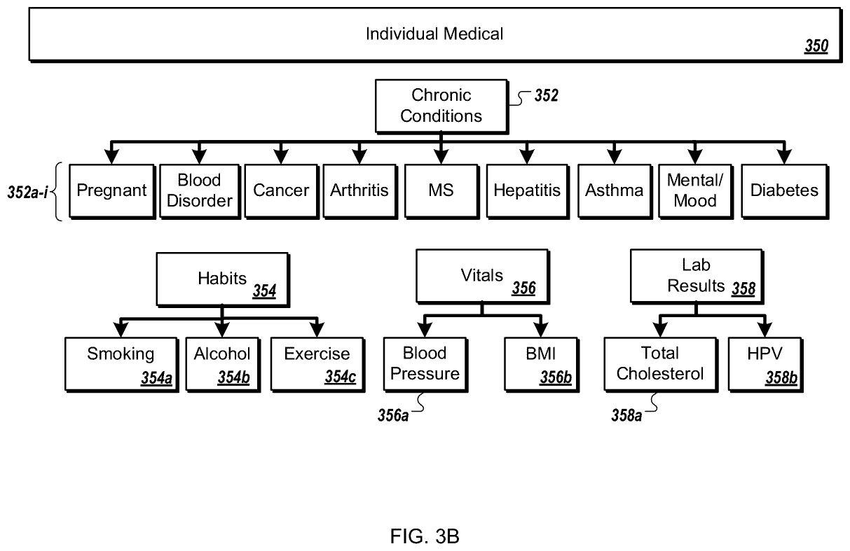

In some implementations, an individual classification engine 116 classifies each member of the target group according to medical condition groupings 152 and/or demographics groupings 154. An example of demographic groupings 154 is presented in FIG. 3A. Further, demographic groupings 154 may be based at least in part on ADI score or ADI grouping, as discussed above. An example of medical condition groupings 152 is presented in FIG. 3B. The medical condition groupings 152, for example, may be based at least in part on the CCSR diagnoses aggregations, as discussed above. The classifications may be used to match each member with similar individuals on a grouping-by-grouping basis (e.g., 100% match for each grouping of N groupings) to establish a control group as similar to the target group as possible.

Turning to FIG. 3A, a set of example individual demographics categorizations 300 includes a gender demographic category 302, an age demographic category 304, an employment status demographic category 306, a geography demographic category 308, an education demographic category 310, and a household income demographic category 312. More or fewer demographic categories may be included. Further, the various demographic categories, in some implementations, may be divided into varying layers of refinement. For example, as illustrated in the age demographic category 304, individuals may be separated into child 304a or adult 304b, then further divided into young and old children, as well as young, middle, and senior adults (e.g., age groupings 0-5 314a, 6-18 314b, 19-23 314c, 24-65 314d, and 66+ 314e). The age ranges are for illustrative purposes only and can be adjusted based on implementation. Further refinements, for example as illustrated in age ranges 316a-h, may be desired in some embodiments. In illustration, separating infants from toddlers (0-2 316a and 3-5 316b) may be desired due to differing medical requirements for these age ranges. The individual classification engine 116 may classify each member of the target group in accordance with the narrowest sub-category of each demographic category.

Turning to FIG. 3B, a set of example individual medical categorizations 350 includes a chronic conditions category 352, a habits category 354, a vitals category 356, and a laboratory results category 358. In some embodiments, an individual may be categorized in a number of sub-categories under each of the main categories. For example, a given individual may have multiple chronic conditions 352a-i (e.g., pregnancy sub-category 352a, blood disorder sub-category 352b, cancer sub-category 352c, arthritis sub-category 352d, multiple sclerosis sub-category 352e, hepatitis sub-category 352f, asthma sub-category 352g, mental/mood disorder sub-category 352h, and/or diabetes sub-category 352i). Further, certain chronic conditions sub-categories are further refined, in some implementations. For example, the arthritis category may be further refined to rheumatoid arthritis, osteoarthritis, and fibromyalgia (not illustrated). For the chronic conditions sub-categories 352a-i, individuals may be categorized in a yes/no manner (does or does not have this particular chronic condition). In the habits category 354, certain sub-categories may be yes/no (e.g., smoking sub-category 354a and/or alcohol sub-category 354b). Conversely, in other implementations, all sub-categories of the habits category 354 may be divided into sub-levels indicating an amount and/or type of activity in each category (e.g., type of smoking habit, ranges of number of cigarettes per week, ranges of number of alcoholic beverages per week, ranges of number of minutes/hours of exercise per week, type(s) of exercise, etc.). Regarding the vitals category 356, each of the vitals (e.g., a blood pressure sub-category 356a and a BMI sub-category 356b) may be separated into at least two ranges (e.g., a clinically concerning level vs an acceptable level). Other range refinements are possible. Finally, in the laboratory results category 358, certain laboratory results may be classified as positive/negative (e.g., an HPV sub-category 358b) while other laboratory results may be classified in ranges (e.g., total cholesterol 358a classified as normal, borderline, and high, etc.). The individual classification engine 116 may classify each member of the target group in accordance with the narrowest sub-category of each medical category.

Returning to FIG. 1, in some implementations, a control group matching engine 120 matches each member of the target group with at least one individual from a pool of control individuals (e.g., identified by control individual data 164 of the data repository 112, each individual's records linked to individual medical data 148 and individual demographics data 150). The control group matching engine 120, for example, uses the medical condition groupings 152 and the demographics groupings 154 to match each member of the target group with at least one individual of the pool of control individuals so that the matched control individual is 100% matched to the target member. The pool of control individuals, for example, may be derived from one or more patient research data sources 105. The pool of control individuals may include tens of thousands, hundreds of thousands, or over a million individuals for matching with the target group. In an illustrative embodiment, the pool of control individuals used may include over 10 million individuals throughout the United States for matching with a target group having a nationally distributed membership.

In an illustrative example, using the individual demographics groupings 300 of FIG. 3A, each target member may be matched to at least one individual having the same gender 302, age range 316 membership, employment status 306 membership, borough or municipality 308e of residence, education 310 level, and household income 312 bracket. Further to the illustration, using the individual medical groupings 350 of FIG. 3B, the matched individuals and the target member will share the same chronic conditions 352, habits 354, vitals 356, and lab results 358. Additionally, in some embodiments, to most closely match medical status between the given target member and the one or more matched individuals, the control group matching engine 120 may match by those individuals having only the same chronic conditions as the target member of the sub-categories of chronic conditions 352a-i. A similar “positive and negative” match may be confirmed for the habits category 354, the vitals category 356, and/or the laboratory results category 358. Since some combinations of chronic conditions may be significantly rare such that exact matches are unlikely, in some embodiments, one or more individuals may be removed from the target group due to lack of an adequate match. For example, to avoid differences in outcomes related to co-morbidities, it may be preferable to remove these unmatched individuals rather than proceeding with a partial match. The control group matching engine 120, for example, may endeavor to match over 99%, at least 99.5%, or preferably 99.8% of the individuals of the target group. In performing this matching, in one example, the geographical range may be expanded to match individuals having rare combinations of chronic conditions. In another example, the age range may be broadened, in certain circumstances, to match individuals with rare combinations of chronic conditions. Other matching techniques are possible.

In some implementations, the members of the target group may also be included in the individuals whose data is analyzed for developing the control group. For example, externally licensed de-identified data may be sourced from millions of records, including records belonging to members of the target group. To avoid matching individuals with themselves, in some embodiments, the control group matching engine 120 may exclude matches as being “too similar” to a target member. In an illustrative example, a potential match demonstrating a same age, location (e.g., zip code or partial zip code), gender, and similar claims submitted in a similar timeframe (e.g., similar month of the year or block of three months, etc.) may be rejected as potentially being the same person.

In some implementations, the control group matching engine 120 matches at least N individuals to each member of the target group to generate a control group that is N times larger than the target group. In some implementations, this allows for any outlier data within the control group (e.g., based upon unusual medical conditions not captured through the matching process) to be mitigated through obtaining data regarding N additional individuals. In another example, the control group matching engine 120 may match another N individuals in case adequate outcome data 158, claims data 146, and/or prescriptions data 142 is not available for a given matched individual. For example, the given matched individual may have recently relocated such that, if the client 104 later desires a follow-on analysis for a next year, one or more matched individuals are still available (e.g., not geographically relocated, not deceased, still having outcome data 158, claims data 146, and/or prescriptions data 142 within the system). In some examples, each target group member may be matched to at least two, three, or up to five control individuals.

The control group matching engine 120, in some implementations, identifies matches based upon historic data availability for at least a threshold period of time. In one example, the threshold period of time may be for a length of time prior to and after an event being evaluated in the target group. The event, for example, may include a change in insurance coverage to the target group, admittance of the target group members to a health program, participation by the target group members in a health study, or participation by the target group members in a clinical trial. In an illustrative example, the event may be addition of a new insurance vendor program, and the time period may be at least one year prior to addition of the new insurance vendor program and at least one year after the addition of the new insurance vendor program. Other threshold timeframes can include, in some examples, at least six months prior to an event and at least three years after the event, at least two years prior to the event and two years after the event, or one to two years prior to the event and at least eighteen months after the event. In another example, beyond simply matching on a set of chronic conditions, each match may be required to have exhibited each chronic condition (e.g., based upon analysis of claims data identifying treatment for the condition, etc.) a period of time. The period of time, in one example, is a set threshold such as, in some examples, a minimum of six months, a minimum of eight months, or a minimum of a year. In another example, the period of time (or a second period of time) matches chronic conditions based upon length of time exhibited. In illustration, if the target member has been dealing with a chronic progressive condition for over five years, a match that has only been treating the chronic condition for seven months may not have the same medical requirements or expense in handling than the later stage patient having the progressive condition.

If no match for a certain member exists, in some implementations, the control group matching engine 120 widens one or more of the medical condition categories or the demographic categories. For example, using the illustration of FIG. 3A, if no matching individual exists (or, alternatively, fewer than N desired matching individuals exist) within the same borough or municipality 308e, the control group matching engine 120 may seek to identify one or more matching individuals sharing the same city 308d with the given member of the target group. Whether or not to widen the pool of candidates, in some embodiments, may depend upon parameters submitted by the requesting client 104. For example, the requesting client 104 may desire tighter controls on matching of certain chronic condition categories 352 but may be less interested in refined age categories 316a-h. Additionally, in some embodiments, refinement of category may vary from member to member. For example, residents of Manhattan may be matched by borough/municipality 308e due to a large number of potential matching candidates within the pool of individuals, while residents of Montana may be initially matched on city 308d or even county/metro region 308c due to the much smaller populations. In some examples, geographic region may be specified by metropolitan statistical area (MSA), combined statistical area (CSA), zip code, or partial zip code (e.g., first three numbers, first four numbers, etc.). Rather than organizing locations of individuals by geographic coding, in some embodiments, individuals in rural areas may be aggregated by reference of closest major hospital. Certain broadening of categories for search purposes, in some embodiments, may require manual approval. For example, the control group matching engine 120 may cause a notification to be relayed to a user prior to performing further matching analysis on a given individual.

After widening one or more medical condition categories and/or demographics categories, if no match is located, in some embodiments, the given member may be removed from the analysis of the target group. For example, a given member may have a very unusual combination of chronic medical conditions or other rare health conditions that is not readily identified within a larger population. Since this individual will have specific unique medical needs in view of the larger group, to avoid any skew of metrics, the individual may be removed from further analysis by the control group matching engine 120. Further, the control group matching engine 120 may report any unmatched members to a user of the health care efficiency analysis platform 102 along with a reason for the lack of match (e.g., no individual in the pool of individuals matched asthma plus multiple sclerosis plus a blood disorder within the demographics categories of the member of the target group).

In some implementations, the control group matching engine 120 stores the matches as control group matches 156 in the data repository 112. A data archival engine 128 may be used to store the control group matches 156. For example, the control group matches 156 may be maintained in long-term storage so that the health care efficiency analysis platform 102 may run analytics for the target group again on a later date for a subsequent time period. In this manner, the health care efficiency analysis platform 102 may track outcomes over time.

In some implementations, an outcomes analysis engine 122 gathers claims data 146, prescriptions data 142, and/or outcome data 158 regarding each of the members of the target group and the control group to establish initial outcomes for each individual. Some claims data 146 may be obtained from the claims data source(s) 106, and some prescriptions data 142 may be obtained from the medical record source(s) 108. Further, some claims data 146, prescriptions data 142, and/or outcome data 158 may be obtained from the benefits coverage source(s) 110. For example, the client 104 may be a benefits organization which supplies expenditures, claims, and/or prescription information for the members of the target group to the health care efficiency analysis platform 102. In some embodiments, the third-party data collection engine 118 may gather the claims data 146, prescriptions data 142, and/or outcome data 158 from the various third-party sources (e.g., claims data source(s) 106, medical record source(s) 108 and/or benefits coverage source(s) 110).

The initial outcomes calculated by the outcomes analysis engine 122, in some implementations, can include total allowed medical efforts, in particular medical costs as one example of medical efforts, per individual (e.g., target group member or control group member), total allowed pharmacy costs per individual, cost of inpatient procedures per individual, cost of outpatient procedures per individual, cost of outpatient professional services per individual, cost of generic medications per individual, cost of brand-name medications per individual, and/or cost of specialty medications per individual. In some implementations, the initial outcomes include quality of care metrics such as, in some examples, correlations between medications prescribed and medications actually filled (e.g., evidence of potential rationing of prescription medications) and/or wellness visit metrics (e.g., whether annual check-ups are performed, whether annual blood screenings are performed, whether age-based annual cancer screenings such as mammograms, colonoscopies, and/or prostrate screenings have been performed, etc.). Many other metrics are possible. The outcomes analysis engine 122 may store the initial metrics data in the data repository 112 as analytics metrics 168. In some embodiments, a data archival engine 128 stores the analytics metrics 168. For example, the analytics metrics 168 may be maintained in long-term storage for later comparison to metrics derived from data collected in a subsequent time frame. Further, for example in embodiments targeting analysis of a clinical trial or post-release statistical gathering for a new medical device, procedure, or medication, the outcomes analysis engine 122 may derive adverse reaction and/or mortality data 144 regarding negative effects to individuals that may be attributed to the new medical device, procedure, or medication.

The outcomes analysis engine 122, in some implementations, combines initial metrics data for each of the N matches to each target group member to obtain control metrics data for comparison to the metrics data of the target group member. In some embodiments, the initial metrics data for the N matches is averaged across the matches. In other embodiments, the initial metrics data may be weighted based upon closeness of match. For example, each matching candidate (e.g., 1 through N) may be correspondingly ranked in order of closeness to the target member. The initial metrics data of the closest matched control individual (e.g., highest ranking of the N matches) may be weighted at greater than 1, while the most dissimilar matched control individual (e.g., lowest ranking of the N matches) may be weighted at less than 1.

In some implementations, a benchmark metrics engine 124 uses the analytics metrics 168 to derive benchmark metrics 166. The benchmark metrics engine 124 may generate metrics regarding the entire target group and the entire control group, as well as sub-sets of each. For example, metrics can be generated based on demographics categories and/or sub-categories (e.g., as illustrated in the example demographic groupings 300 of FIG. 3A), medical condition categories and/or sub-categories (e.g., as illustrated in the example medical condition groupings 350 of FIG. 3B), or other demographics and/or medical conditions (e.g., as derived from the individual medical data 148, individual demographics 150, member data 162, and/or control individual data 164). In a particular example, metrics may be calculated based on count of chronic medical conditions (e.g., individuals with more than 2 chronic medical conditions, more than 3 chronic medical conditions, etc.). The benchmark metrics engine 124 may store the benchmark metrics in the data repository 112 as benchmark metrics 166. In some embodiments, the data archival engine 128 stores the benchmark metrics 166. For example, the benchmark metrics 166 may be maintained in long-term storage for later comparison to metrics derived from data collected in a subsequent time frame.

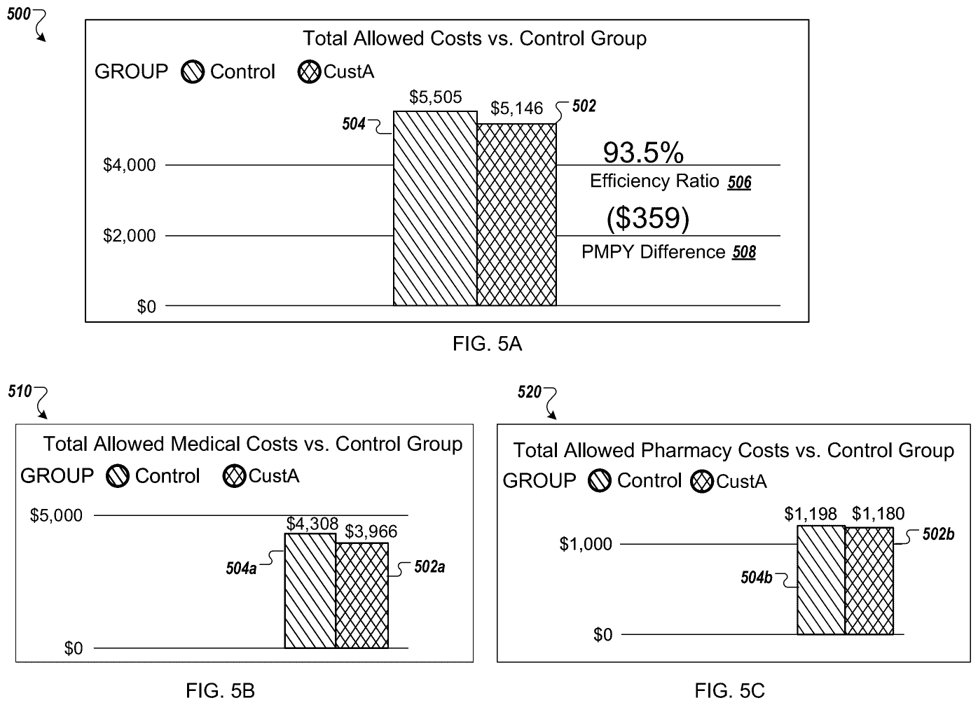

In some implementations, a report generation engine 126 obtains the benchmark metrics 166 and prepares graphic output comparing target group metrics and control group metrics. The graphic output, for example, may include tables, graphs, charts, and/or automatically generated brief commentary regarding results of the analysis. The report generation engine 126 may prepare the graphic output in one or more formats such as, in some examples, a web-enabled format for access via a web browser or Internet-enabled user portal, a document format such as a Microsoft Word document or Adobe PDF document, and/or a spreadsheet format such as a Microsoft Excel spreadsheet. If prepared in a web-enabled format, in some embodiments, the graphic output may be interactive, allowing for drill-down access to greater levels of detail. The report generation engine 126 may enable delivery of the report to a user. Delivery, in some examples, may include email, printing, or causing presentation to a remote display device. Example screen shots of report graphics are illustrated in FIGS. 5A-5C, FIG. 6, FIG. 7, and FIGS. 8A-8C, described in further detail below.

If data is available for multiple time frames, in some implementations, a progress analysis engine 130 analyzes the analytics metrics 168 and/or benchmark metrics 166 as gathered over the multiple timeframes to calculate progress metrics 170. The progress metrics 170, in some examples, may include a percentage change in cost over time (e.g., for any of the cost metrics discussed above), total change in cost over time, percentage change in chronic conditions in members of each of the target group and the control group over time, percentage change in adverse reactions and/or mortality metrics over time, and/or total change in adverse reactions and/or mortality metrics over time. In some embodiments, the data archival engine 128 stores the progress metrics 170. For example, the progress metrics 170 may be maintained in long-term storage for later comparison to metrics derived from data collected in a subsequent time frame (e.g., quarterly metrics over first year vs. quarterly metrics over second year, etc.).

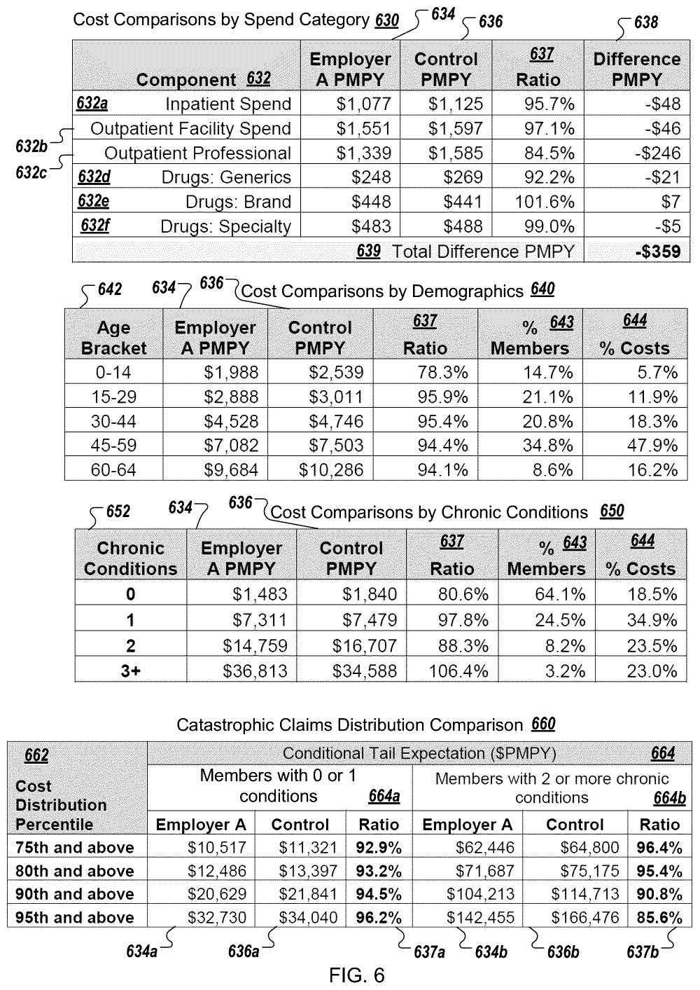

The progress metrics 170 produced by the progress analysis engine 130, in some implementations, are obtained by the report generation engine 126 to generate graphic output comparing target group metrics and control group metrics over time. Example screen shots of report graphics for progress metrics are illustrated in FIG. 6, described in further detail below.

In some implementations, the health care efficiency analysis platform 102 supports user-customizable control group selection, for example through selective identification of categories, sub-categories, ranges, and/or other matching factors used to match members of the target group with one or more members of the control population. Further, the health care efficiency analysis platform 102 may provide a user-customizable number of matches to produce for each member of the target group. Further, certain report metrics (e.g., analytics metrics 168, benchmark metrics 166, and/or progress metrics 170) may be user-configurable to customize report outputs to user needs. In this manner, the health care efficiency analysis platform 102 may be accessed by clients 104 to generate analysis highlighting key factors of interest to the individual client. The key factors, for illustrative purposes, may include increase in member exercise, reduction in expression of chronic conditions among members, lowering of total cholesterol among members, and/or increase in vaccination among the member population.

A matching factor selection engine 132, in some implementations, provides clients 104 with the opportunity to select categories and/or sub-categories within a comprehensive list of medical condition groupings 152 and/or demographic groupings 154. Further, the matching factor selection engine 132 may provide the clients 104 with the ability to set customized ranges, thresholds, or other groupings for particular categories and/or sub-categories. For example, the age sub-categories 314a-e or 316a-h may be user-customizable to target key demographic age ranges relevant to the client 104. The individual classification engine 116 may classify the individuals of the control population pool as well as the members of the target group according to the customized medical condition groupings 152 and/or demographic groupings 154 to assist the control group matching engine 120 in matching each target group member with one or more control individuals.

In some implementations, a matching factor weightings engine 134 provides clients 104 with the opportunity prioritize categories and/or sub-categories within a comprehensive list of medical condition groupings 152 and/or demographic groupings 154. For example, the client may select that matching on particular chronic medical conditions is required, while matching geographic regions is desirable but not necessary (e.g., the system can default to matching any individual in the country with the same chronic medical conditions as a given target group member). In some embodiments, further refinements of matching prioritization are possible (e.g., necessary, preferred, not concerned). The control group matching engine 120 may use the weightings of the medical condition groupings 152 and/or the demographic groupings 154 to perform matching analysis of the target member group to the pool of control individuals.

In some implementations, a dimensionality reduction engine 136 automatically reduces medical factors, such as condition identifiers and/or prescription identifiers, to group same or similar elements to assist in matching. For example, a same prescription drug may be provided in multiple forms and/or doses as well as by multiple manufacturers, such that a single type of antihistamine, for example, may be offered as over a dozen options. Similarly, condition identifiers, such as International Statistical Classification of Diseases and Related Health Problems (ICD) codes, a global standard released by the World Health Organization, may include tens or even hundreds of codes relevant to a particular chronic medical condition, such as diabetes or multiple sclerosis. Beginning with thousands or tens of thousands of condition identifiers associated with the individuals of a member population, the dimensionality reduction engine 136 may reduce the corresponding conditions to a corresponding hundreds of conditions or, in other words, the initial conditions may be reduced by about a factor of 10 to a factor of 100. In some examples, the dimensionality reduction engine 136 may apply statistical analysis and/or machine learning classifiers to reduce the initial pool of condition identifiers. Further, the dimensionality reduction engine 136 may select from the resultant tens or hundreds of conditions a subset of conditions for analysis.

The dimensionality reduction engine 136, in some implementations, reduces conditions in part by filtering out at least a portion of codes related to diagnostic services, such as laboratory service codes. For example, many diagnostic services relate to determining whether or not a patient has a condition, while those patients with the condition will receive additional services that relate mostly or solely to those with the given condition. In another example, identifiers corresponding to various condition types (e.g., cancer, cardiovascular disease, etc.) may be organized into hierarchical groupings, where each layer of the hierarchy includes one or more condition sub-types. Further, certain sub-types within a hierarchical layer may be weighted or promoted in impact in comparison to other sub-types for purposes of matching the members with the pool of individuals. In illustration, mental illness may include a hierarchical layer including substance abuse, depression, anxiety, adjustment disorder, and attention-deficit/hyperactivity disorder (ADHD). Within this layer, sub-type depression, being more severe, long-term, and threatening to a patient's health, may be weighted, promoted, or ranked higher than adjustment disorder.

In some implementations, the dimensionality reduction engine 136 prioritizes the resultant conditions to identify a subset of conditions that have the greatest impact on the analysis being performed by the health care efficiency analysis platform 102. For example, the dimensionality reduction engine 136 may identify up to about a number X conditions (e.g., ten, twenty, thirty, etc.) from all conditions identified. The number X may vary based on a total number of members in the target member group such that larger populations (e.g., hundreds of thousands or millions of members) may be analyzed based on a larger number of conditions, while small populations (e.g., hundreds or thousands) may be analyzed based on a smaller number of conditions. In the circumstance of analyzing outcomes related to the member population, for example, the dimensionality reduction engine 136 may identify the X most impactful to the cost profile of the organization. In another illustration, the dimensionality reduction engine 136 may identify the most impactful therapies within the member population to health outcomes, for example based on reviewing engagement data related to members attending therapy sessions. The data, in an illustrative example, may be derived from Employee Assistance Program (EAP) data and/or Licensed Alcohol and Drug Counselor (LADC) data. Priorities may differ based on the type of analysis (e.g., pharmaceutical vs. medical and pharmaceutical). For example, for some conditions pharmaceutical costs greatly outweigh medical services costs, such as HIV therapy. Priorities may differ within the member population, such as, in some examples, different age groups within the member population, different geographical locations of the members, genders of the members, and/or other demographic refinements.

Because trends in medical conditions may differ within groupings of members in the population, in some embodiments, the dimensionality reduction engine 136 iteratively identifies the conditions having the greatest impact in each of a number of demographic groupings. In illustration, for each of a set of age brackets, for each gender within each age bracket, and for each gender living in each geographic region of a set of geographic regions within each age bracket, the dimensionality reduction engine 136 may identify the most impactful conditions for each grouping. Further, geographic refinements may be iteratively analyzed. In the circumstance of the United States, for example, a member population may be broken down by state, further by county, then by city. The level of geographic refinement, for example, may depend in part on the size of the member population and/or the population density of each region. For example, a densely populated metropolitan region such as New York City may be broken into boroughs and even further into a census block level.

Once the most impactful conditions within each member grouping have been determined, in some embodiments, the dimensionality reduction engine 136 selects a final number of most impactful conditions affecting people in both groups. The engine uses statistical analyses to find the subset of conditions (usually about 15-25 conditions) which explain the most variation (usually above 90% of the variation) in healthcare expenditures.

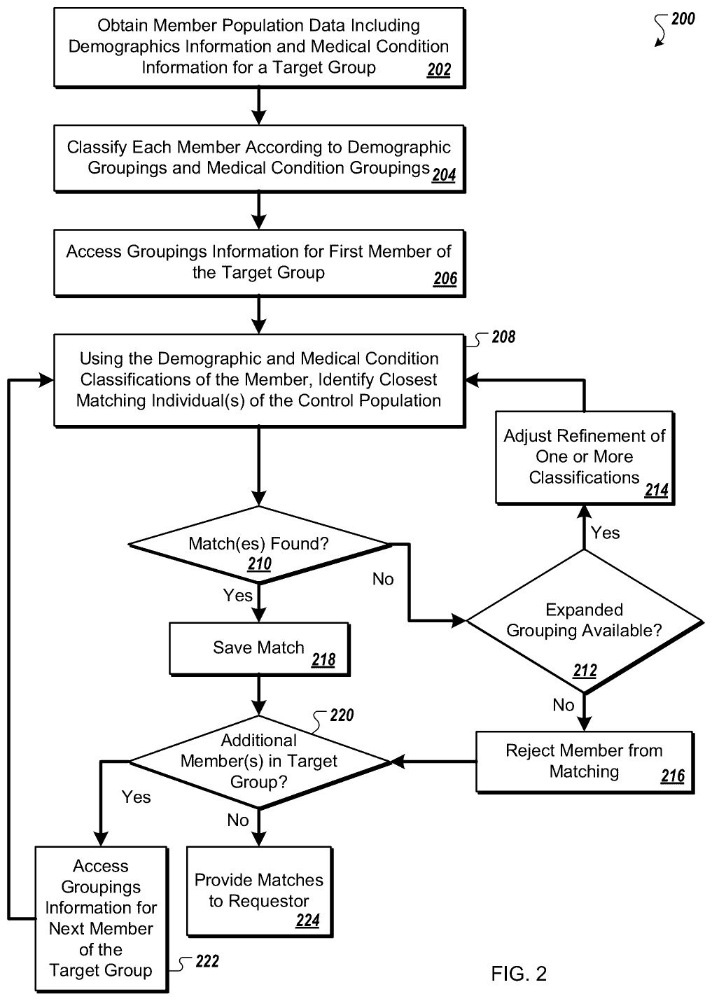

Turning to FIG. 2, a flow chart illustrates an example method 200 for matching control individuals to each member of a target group. The method 200, for example, may be performed by the health care efficiency analysis platform 102 of FIG. 1.

In some implementations, the method 200 begins with obtaining member population data including demographics information and medical condition information for a target group (202). The medical information, in some embodiments, includes patient invoices and/or insurance reimbursement information including medical codes corresponding to prescriptions and/or services. Each code may be individually indicative of one or more medical conditions. In an illustrative example, diabetes, in general, may be represented by hundreds of codes or more, while a portion of these codes may be indicative of a particular type of diabetes. The demographics information may include patient information, employee record information, and/or resident information (e.g., in an assisted living facility or long-term care facility, etc.). The demographic information may include environmental factors such as, in some examples, ADI score, regional disease/disorder propensities, and/or hazardous condition exposure (e.g., water quality, air quality, etc.). The demographics information may have been collected at least in part on behalf of an organization conducting clinical trials or other testing or patient monitoring programs. In some embodiments, the demographics information may be entered into the system personally by at least a portion of the members.

In some implementations, each member of the target group is classified according to a number of demographic groupings and a number of medical condition groupings (204). The demographic groupings may include, in some examples, gender groupings, age groupings (e.g., infant, juvenile, teen, adult, senior, and/or age ranges), BMI groupings, geographic region groupings (e.g., city, county, state, province, etc.), and/or environmental groupings (e.g., ADI score categories, environmental risk factor categories, etc.). Further, in some embodiments, the demographic groupings include behaviors such as, in some examples, smoking, alcohol use, recreational drug use, and/or job activity level (e.g., sedentary, physically strenuous, etc.). The medical condition groupings may include a number of diseases and/or conditions commonly resulting in long-term and/or expensive treatments or therapies. Further, the medical condition groupings may include a number of diseases and/or conditions commonly resulting in later complications, conditions, or diseases. Some groupings may be overlapping. Some groupings may be encompassed by other groupings, such as both Type I and Type II diabetes being encompassed by a diabetes grouping. The groupings in some embodiments, are determined in part using machine learning classification. The classification, for example, may be performed by the individual classification engine 116 of FIG. 1.

In some implementations, groupings information is accessed for a first member of the target group (206). The groupings information, including demographics groupings and medical groupings, may be stored within a computer-readable medium, such as cloud storage or a relational database. In some implementations, the information is stored in a secure, de-identified, and/or encrypted manner to protect the groupings from being matched to an identification of the first member of the target group. The groupings information, for example, may be stored in the data repository 112 of FIG. 1.

In some implementations, one or more closest matching control individuals of a control population to the first member of the target group are identified using the demographic and medical condition classifications (208). The matching, for example, may be performed as described in relation to the control group matching engine 120 of FIG. 1.

In some implementations, if a match (or, in some embodiments, less than a threshold number of matches) is found (210), it is determined whether an expanded grouping is available for one or more unmatched variables (212). The matching, for example, may be performed first to capture more narrow groupings (e.g., type and stage of cancer diagnosis) and, if insufficient matches are found, the grouping may be broadened (e.g., “late stage” cancer may encompass both stage III and stage IV) to identify matching control individual(s).

If an expanded grouping is available (212), in some implementations, a grouping of one or more demographic or medical condition classifications are expanded (214). Conversely, if no expanded grouping is available (212), in some implementations, the first member of the target group is rejected from the matching process (216). As discussed above in relation to FIG. 1, not all individuals may be matched to corresponding control group members.

Returning to step 210, in some implementations, if the desired number of matches is found (210), the match(es) are saved (218). Each match, for example, may be saved as control group matches 156 in the data repository 112, as described in relation to FIG. 1.

In some implementations, while additional members of the target group exist (220), the groupings information for the next member of the target group are accessed (222) and process returns to using the demographic and medical condition classifications of the member to identify the closest matching individual(s) of the control population (208).

Once no additional members of the target group are left unmatched (220), in some implementations, the matches are provided to a requestor (224). The requestor may be an end user, such as an individual interacting with one of the client systems 104 of FIG. 1. In other embodiments, the requestor is another software engine, such as the report generation engine 126 of FIG. 1.

The method 200 is illustrated as a particular set of operations. In other embodiments, more or fewer operations may be included in the method 200. For example, in some embodiments, rather than broadening a grouping, only a single layer of groupings may be applied to perform matching. In another example, in addition to grouping members by demographic and medical condition groupings, the members may be grouped by a third category, such as health maintenance program, alternative medicine regimen, or other ongoing therapeutic intervention/treatment. In an illustrative example, alternative medicine programs may include therapeutic massage, chiropractic adjustments, and/or acupuncture treatments. In another example, an ongoing therapeutic intervention may include a specialized diet or nutrient supplement.

Although described in relation to a particular series of operations, in other embodiments, one or more steps may be performed in a different order and/or in parallel. For example, matches of multiple members of the target group may be performed in parallel. Other modifications of the method 200 are possible while remaining in the spirit and scope of the process.

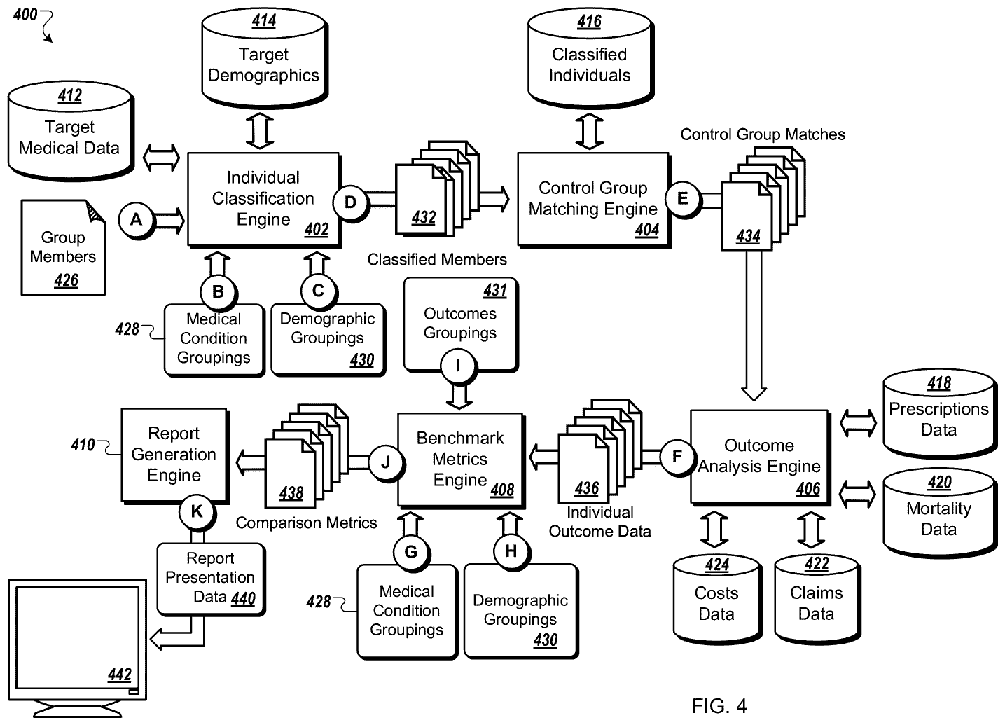

Turning to FIG. 4, an operational flow diagram illustrates an example process 400 for automatically generating a report presenting comparison metrics between outcomes of a members of a target group and outcomes of members of an automatically generated control group. The process 400, for example, may be performed by the health care efficiency analysis platform 102 of FIG. 1.

The process 400, in some implementations, begins with obtaining, by an individual classification engine 402, information regarding a set of group members 426. The group members 426, for example, may cross-reference, link to, or otherwise match up with medical conditions collected in target medical data 412 and target demographics data 414 regarding each individual of the group. The information, in some embodiments, is included in the individual medical data 148 and the individual demographics 150 of FIG. 1.

In some implementations, the individual classification engine 402 receives indication of medical condition groupings 428 and demographic groupings 430 for categorizing the demographic and medical condition information of the group members 426. As described in relation to FIG. 1, for example, the demographic groupings 430 may be the demographic groupings 154 of FIG. 1.

The individual classification engine 402, in some implementations, filters the target medical data 412 and target demographics data 414 using the medical condition groupings 428 and demographic groupings 430 to determine a set of classifications for each of the group members 426. In other words, based upon an individual's medical data 412, each group member may include classifications within one or more chronic conditions, such as the chronic conditions 352 illustrated in FIG. 3B. Further, each group member may include classifications regarding habits 354, vitals 356, and/or laboratory results 358. The number of classifications per individual group member will likely vary, in preferred embodiments, due to differences in individuals' health status. In some implementations, the individual classification engine 402 generates a set of member classifications 432 for use by a control group matching engine 404.

The control group matching engine 404, in some implementations, matches each of the group members 426 to members of a classified population (classified individuals 416) based on the set of member classifications 432 (e.g., medical condition groupings and the demographic groupings). For example, the control group matching engine 404 may determine matches in a manner similar to that of the method 200, described in relation to FIG. 2.

In some embodiments, the classified individuals 416 are pre-classified according to the medical condition groupings 428 and the demographic groupings 430 (e.g., set groupings). In embodiments where the medical condition groupings 428 and/or demographic groupings 430 are customizable, the individual classification engine 402 may also classify the control population to generate the classified individuals 416, for example, based on control group medical data and/or demographics.

The control group matching engine 404, in some implementations, generates a set of control group matches 434, identifying matches between at least a portion of the target group members 426 and classified individuals 416. The control group matches may include identifiers of control group individuals matching individual group members 426. The control group matches 434, further, may indicate matched groupings per control group individual (e.g., matched based on A, B, and C demographics as well as X, Y, and Z medical conditions). The control group matches 434 may be used by an outcome analysis engine 406 to analyze outcome over time for the control group matches 434.

In some implementations, the outcome analysis engine 406 accesses, for each individual in the control group matches 434, data related to outcomes in treatment efficacy, insurance costs or other metrics associated with the medical condition(s) of the individuals 434. For example, the outcome analysis engine 406 may access prescription data 418, mortality data 420, claims data 422, and/or costs data 424 spanning a timeframe. The timeframe may represent, in some examples, six months, one year, eighteen months, or two years or more. The timeframe, for example, may be selected based upon the medical conditions being monitored. For example, in a smoking cessation program, the timeframe may be shorter (e.g., six months or a year) in comparison to analyzing mortality rates for certain cancers which may require synthesizing years of data. In an example related to insurance usage, the prescriptions data 418, claims data 422, and/or costs data 424 may be analyzed to estimate medical claim types and/or medical claim costs related to the control group matches 434. The outcome analysis engine 406 generates individual outcome data 436 representing outcomes (efforts, costs, procedures, recoveries, deaths, prescription usage, etc.) for the control group matches 434.

A benchmark metrics engine 408, in some implementations, calculates comparison metrics 438 comparing groupings of the individual outcome data 436. For example, the control group matches 434 may be grouped by one or more demographic groupings 430, outcomes groupings 431, and/or medical condition groupings 428. Groupings, in some examples, can include age ranges, gender, number of chronic conditions or co-morbidities, and/or geographic region. In some examples, the comparison metrics 438 may compare insurance costs, number of prescriptions, number of hospital visits, mortality rate, and/or recovery/remission rate across various groupings. The groupings, for example, may include the groupings represented in FIG. 3A and FIG. 3B. The benchmark metrics engine 408, in some embodiments, ranks or organizes outcomes in the individual outcome data 436 by frequency or prevalence. For example, the benchmark metrics engine 408 may identify the most common chronic conditions across the group member 426.

The benchmark metrics engine 408, in some implementations, is used to forecast outcomes of the group members 426 based on outcomes of the control group matches 434. In further implementations, the benchmark metrics engine 408 is used as a comparison tool to compare outcomes (e.g., efforts, costs, procedures, recoveries, deaths, prescription usage, etc.) of the group members 426 to a control member population to determine whether policies, treatments, and/or therapies applied to the group members 426 made a positive (or negative) difference in comparison to a general population. In this circumstance, the comparison metrics 438 calculated by the benchmark metrics engine 408 include comparison metrics of control group 434 outcomes to group member 426 outcomes.

In some implementations, a report generation engine 410 transforms the comparison metrics 438 into report presentation data 440 for generating a graphical report. The report may be printed and/or presented at a display 442. For example, the report presentation data 440 may be configured to present an interactive review of the data such that a reviewer may drill down into the comparison metrics (e.g., dig into refined groupings). The report generation engine 410 may generate graphical comparisons of the comparison metrics 438 such as charts, tables, and/or graphs of information. Examples of report screen shots are presented in FIG. 5A through FIG. 5C and FIG. 6. As will be described in further detail below, the comparison metrics 438, when presented in various levels of refinement and in a number of different graphic outputs, can be used to provide an end user with a detailed breakdown of risks, anticipated costs, and/or estimated benefits derived from programs applied by the user's organization, turning what, in the prior art, would be subjective analysis into objective, actionable data.

Turning to FIG. 5A through FIG. 5C, a set of bar graphs 500, 510, and 520 illustrate total allowed costs 502 of the member population (“CustA”) in comparison to a control group 504. FIG. 5A illustrates total costs, while FIG. 5B and FIG. 5C break the total costs down into total allowed medical costs 502a, 504a and total allowed pharmacy costs 502b, 504b, respectively. The allowed costs, in the illustrated example, are the costs authorized by the insurance plan(s) of both the control group and of the customer CustA and paid by insurance, not considering member deductible, copay, other insurance coverage. In FIG. 5A, a cost efficiency ratio 506 of 93.5% and a per member per year (PMPY) dollar amount difference 508 of $359 are also illustrated.

Turning to FIG. 6, a cost comparison by spend category table 630 further breaks down the medical and pharmaceutical costs into components 632 including inpatient spend 632a, outpatient facility spend 632b, outpatient professional 632c, generic drugs 632d, brand drugs 632e, and specialty drugs 632f. The pharmaceutical treatments and medical services may be categorized, for example, based upon an industry coding standard. For each of the components 632, the table 630 presents an employer PMPY 634, a control group PMPY 636, a ratio 637 of the employer PMPY 634 to control PMPY 636, and a PMPY difference 638 in dollar value. Finally, the cost comparison by spend category table 630 provides a total PMPY difference 639 in dollars across all components 632. The cost comparison by spend category table 630, for example, may identify improvements in costs compared to a control group gained through differentiating the health care benefits and/or wellness program offerings by the employer.

A cost comparison by demographics table 640 presents similar information, including employer PMPY 634, control group PMPY 636, and ratio 637, this time broken down into a series of age brackets 642 (0-14, 15-29, 30-44, 45-59, and 60-64). The cost comparison by demographics table 640 additionally presents a percentage of members 643 per age bracket and a percentage of costs 644 per age bracket. The cost comparison by demographics table 640, for example, may be used to identify the age range of employees that the employer should target in wellness programs to improve health spending.

A cost comparison by chronic conditions table 650 presents the employer PMPY 634, the control group PMPY 636, the ratio 637, the percentage of members 643 and the percentage of costs 644, this time broken down by a total number of chronic conditions 652 per member segment (none, 1, 2, and 3 or more). The cost comparison by chronic conditions table 650, for example, highlights the importance of wellness efforts for avoiding and/or early interventions for curing or diminishing the effects of chronic conditions.

Finally, the user interface of FIG. 6 presents a catastrophic claims distribution comparison table 660 for analyzing catastrophic claims across a member population over a period of time in comparison to the control population. The catastrophic claims distribution comparison table 660 analyzes conditional tail expectation (CTE) 664, quantifying a risk (e.g., anticipated cost) based upon a catastrophic medical event occurring outside of a given probability 662. The catastrophic claims distribution comparison table 660 presents the anticipated costs as employer PMPY 634a, 634b and control group PMPY 636a, 636b, as well as the ratio 637a, 637b in both a first membership group 664a having zero or one chronic medical condition and a second membership group 664b having two or more chronic medical conditions. The catastrophic claims distribution comparison table 660, for example, may present, to an end user, potential worst case scenario medical costs associated with the employer membership in comparison to a control population.

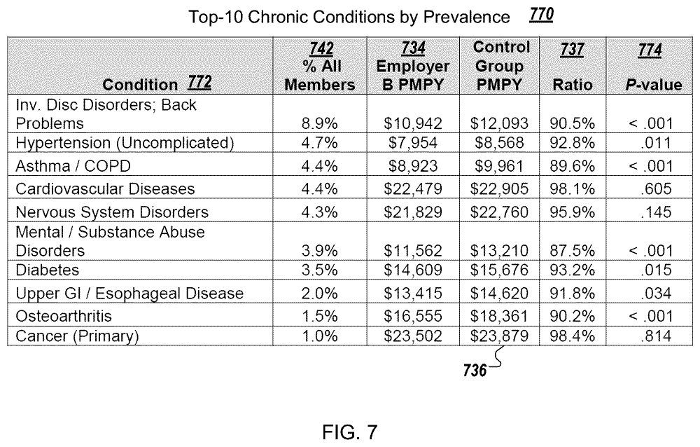

Turning to FIG. 7, a top 10 chronic conditions by prevalence table 770 illustrates the impact of the most common chronic conditions 772 among the control group membership and the employer membership. As illustrated, the ten conditions 772 include disc disorders and back problems, hypertension, asthma/COPD, cardiovascular diseases, nervous system disorders, mental/substance abuse disorders, diabetes, upper GI/esophageal disease, osteoarthritis, and cancer. For each condition, the top 10 chronic conditions by prevalence table 770 presents a percentage of members 742 having the condition, an employer PMPY 734, a control group PMPY 736, a ratio 737 of the employer PMPY 734 to the control group PMPY 736, and a p-value 774. The p-value 774 reflects the chances of finding no difference in costs between the two groups if the analyses were repeated many times (e.g., smaller p-values are better, since they would reflect small chances that the costs are equal to each other). A p-value 774 of five percent or lower, for example, may be considered to represent a significant difference in the PMPY ratio 737. The data presented in the top 10 chronic conditions by prevalence table 770, for example, may illustrate to the end user opportunities for avoiding health issues, such as providing training and tools to safely lift heavy objects, thereby reducing disc disorders and back problems.

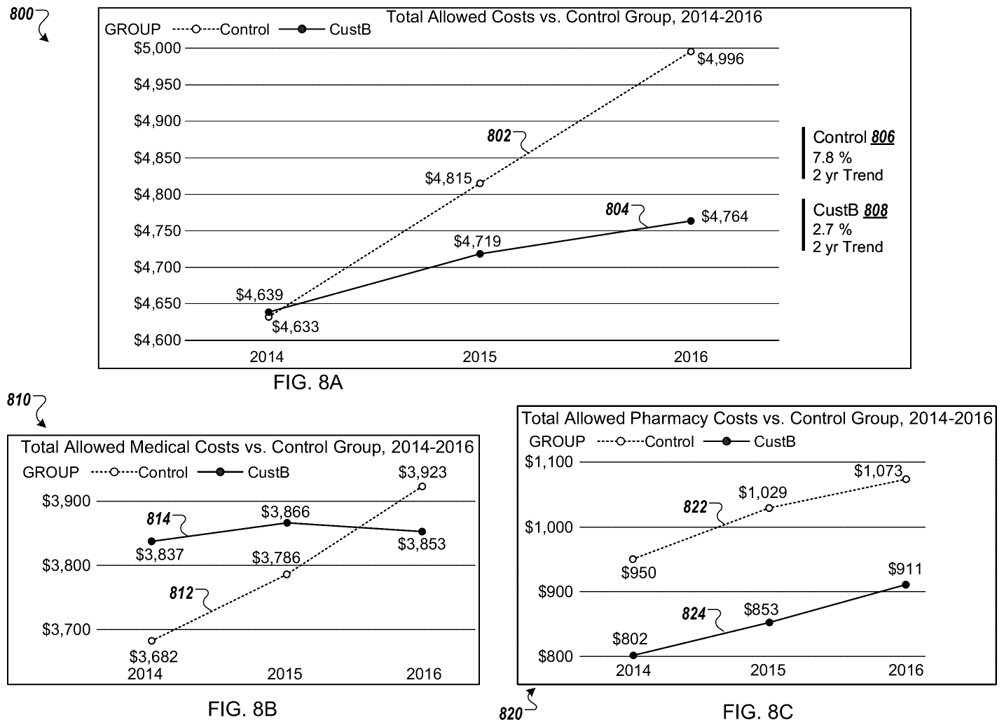

FIGS. 8A through 8C illustrate year-over-year trends in total costs to a customer membership 808 (e.g., 7.8% total increase) in comparison to a control group membership 806 (e.g., 2.7% total increase), as illustrated spanning the years 2014-2016. This output, for example, may present an end user with data establishing proof-of-concept related to a program, intervention, or therapy improving the bottom line by matching members of the test population to a control population, thereby lessening or removing the effects of other factors on the analysis.

Turning to FIG. 8A, a total allowed costs vs. control group graph 800 presents a line graph comparing costs per year in 2014, 2015, and 2016 between a control cost line 802 and a customer cost line 804. As illustrated, year-over-year costs for the customer rose far slower than costs for the control group.

FIG. 8B and FIG. 8C break these costs down, first in a total allowed medical costs vs. control group graph 810 comparing control costs 812 to customer costs 814 and then in a total allowed pharmacy costs vs. control group graph 820 comparing control costs 822 to customer costs 824. As seen in the graphs 810 and 820 of FIG. 8B and FIG. 8C, respectively, medical costs were initially higher for the customer membership in year 2014 but remained fairly stagnant such that the control costs 812 ended up exceeding the customer costs 814 by year 2016. This may be indicative of application of a successful plan to reduce medical costs by the customer. Conversely, although total allowed pharmacy costs for the customer membership began lower than the control group costs in 2014, both rose at a similar rate.

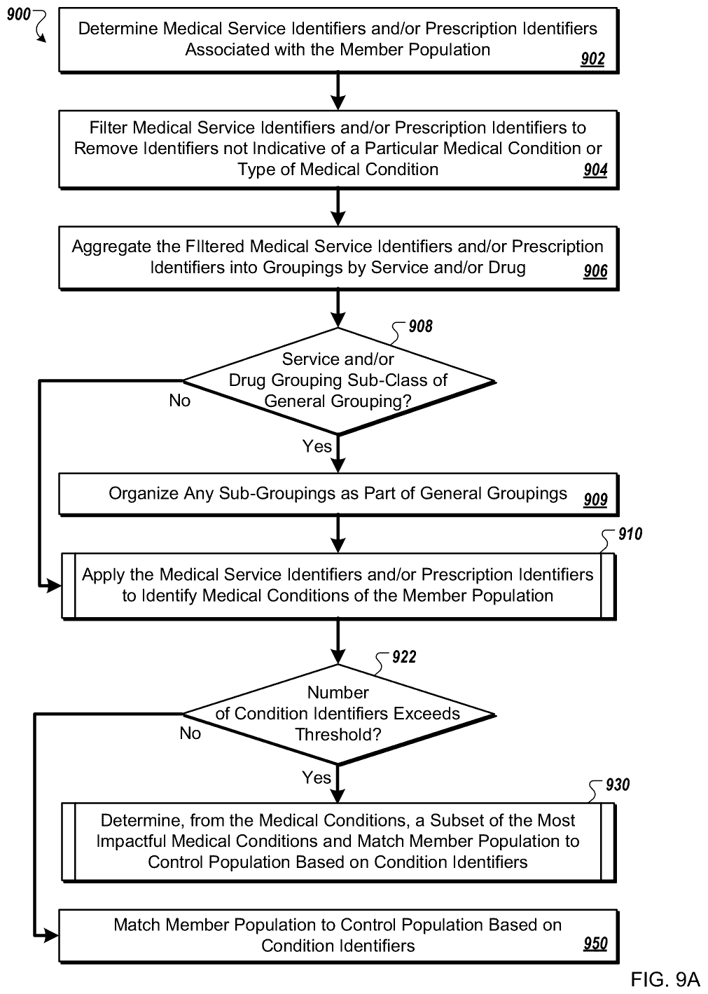

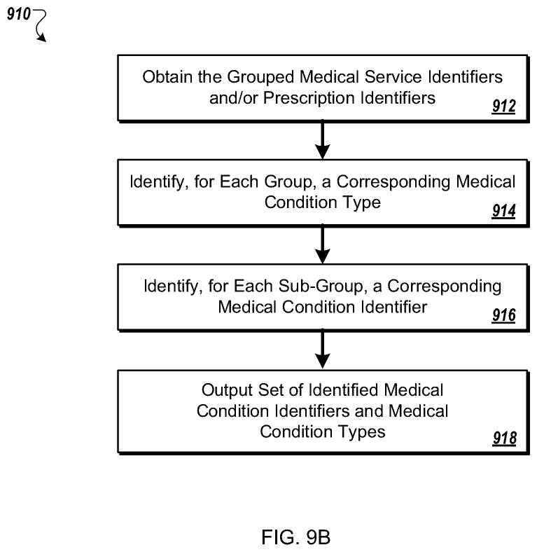

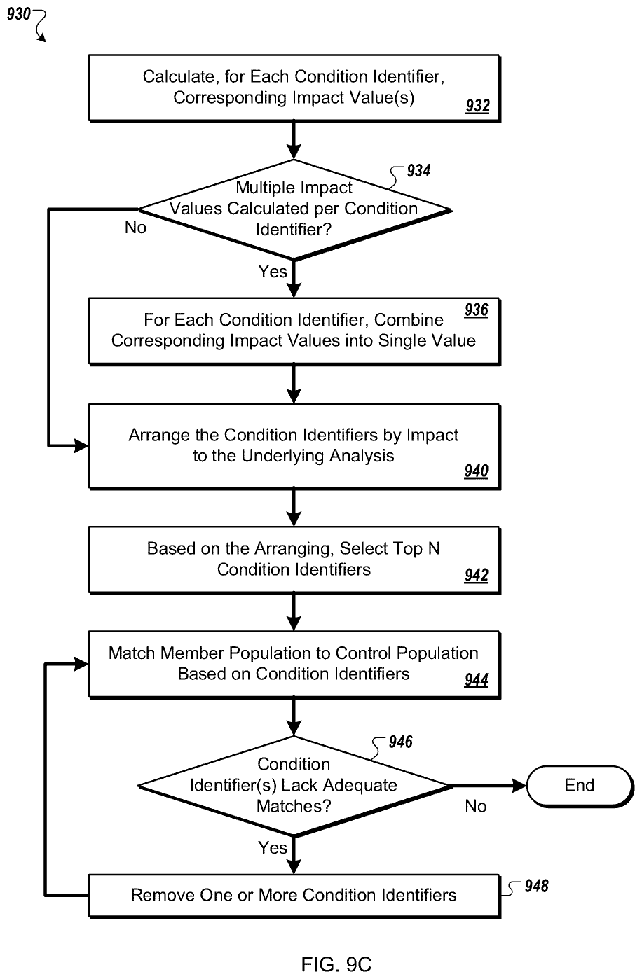

FIGS. 9A through 9C illustrate flow charts of an example method 900 and sub-methods 910 and 930 for identifying features for matching members of a target population with members of a control population. Portions of the method 900 and sub-methods 910 and 930 may be performed, for example, by the matching factor selection engine 132, the matching factor weightings engine 134, and/or the control group matching engine 120, described in relation to FIG. 1.

Turning to FIG. 9A, in some implementations, the method 900 begins with determining condition identifiers and/or prescription identifiers associated with a member population (902). The identifiers, in some examples, may be included in the claims data 146, the individual medical data 148, and/or one or more of the claims data sources 106 or the benefits coverage sources 110, as described in relation to FIG. 1. The identifiers, for example, may include disease identifiers, service identifiers, and/or pharmaceutical identifiers. Examples of identifiers may include the International Statistical Classification of Diseases and Related Health Problems (e.g., ICD-10, ICD 11) disease codes by the World Health Organization (WHO), SNOWMED CT codes defined by the International Health Terminology Standards Development Organisation (SNOMED International of London, UK), or National Drug Codes (NDCs) defined by the U.S. Food and Drug Administration to identify prescription pharmaceuticals.

In some implementations, the medical service identifiers and/or prescription identifiers are filtered to remove identifiers not indicative of a particular medical condition or type of medical condition (904). Diagnostic medical codes indicative of monitoring for potential medical conditions may be removed such as, in some examples, imaging codes and/or laboratory screenings.

In some implementations, the filtered condition identifiers and/or prescription identifiers are aggregated into groupings by condition and/or drug (906). The identifiers may include a prefix or first X alphanumerical characters representing a broader concept. In illustration, using the ICD-10 codes, 150.9 represents unspecified heart failure, 150.X specifies heart failure in general, and 100-199 are reserved for circulatory system diseases in general. In this circumstance, the codes themselves may guide initial groupings. In another example, natural language processing of a code standard, including synonym searches, may be used to automatically group condition identifiers and/or prescription identifiers. In illustration, all codes listing “congestive heart failure” or “CHF” may be grouped together. In a further example, in some embodiments, individual condition identifiers may represent multiple chronic conditions. In illustration, an identifier representing “hypertensive heart and chronic kidney disease” may be parsed into two general conditions of “hypertensive heart disease” and “chronic kidney disease.”

In some embodiments, due to the complexity of arranging tens of thousands of identifiers into groupings, the groupings may be pre-categorized. Pre-classification may be based on machine learning analysis or other statistical analysis. For example, machine learning classifiers may be developed to parse and categorize general types of conditions and/or pharmaceuticals into narrower categories or groupings. A first classifier, for example, may be trained to separate diabetes identifiers by type (e.g., pregnancy-induced diabetes may be separated from preexisting diabetes during pregnancy/post-partum despite their similarity in wording). Further to the example, a second machine learning classifier may be trained to separate types of carcinomas. A same type of machine learning classifier may be developed for each type of identifier if two or more classification systems are analyzed. The machine learning classifiers, in an illustrative example, may be developed as regularized regression decision tree, and/or gradient models to capture the layers of granularity in the condition and/or prescription identifier coding structure.

If one or more given condition groupings and/or drug groupings is a sub-class of a broader or more general grouping (908), in some implementations, the sub-grouping(s) are organized as part of the broader or more general groupings (909). In an effort to determine an appropriate level of specificity (e.g., more than one member of the population with the same condition, not too broad as to incur widely varying costs, etc.), the groupings may be arranged or linked within a hierarchy.

In some implementations, the medical service identifiers and/or prescription identifiers are applied to identify medical conditions of the member population (910). Turning to FIG. 9B, in some implementations, the sub-method 910 begins with obtaining grouped medical service identifiers and/or prescription identifiers (912). The groupings, for example, may be obtained from a database or other data storage.

In some implementations, for each grouping, a corresponding medical condition type is identified (914). The medical condition type, for example, may be identified through a look-up table or other linking that maps the groupings to a medical condition type.

A medical condition identifier, in some implementations, is identified for each sub-group of any group having sub-groups (916). For example, for a medical condition type of diabetes, the sub-group medical condition identifiers may include type I and type II. As with the medical condition type, the medical condition identifiers may be identified via a look-up table or other linking that maps sub-group identifiers to a medical condition identifier.

In some implementations, the set of identified medical condition identifiers and medical condition types are output by the method 910 (918). The medical condition identifiers and medical condition types, for example, may be stored to a data store or passed to a calling software module.

Returning to FIG. 9A, in some implementations, if a total number of groupings of condition identifiers and/or prescription identifiers exceeds a threshold value (922), a sub-set of the most impactful medical conditions is determined (930). Setting a threshold value, for example, may improve the number of successful matches between the member group and the control group by limiting the conditions applied to matching. Further, a smaller threshold value may speed the operation of performing the matching. In another example, a smaller threshold value may assist in better pinpointing the major factors contributing to the impact of the underlying analysis (e.g., mortality rate, health care efforts, costs, facility utilization, therapy completion, etc.). In some embodiments, the threshold is a set value or user-selected value, such as, in some examples, twenty, thirty, or fifty identifiers. In some embodiments, the threshold is based at least part on a median, average, or percentile portion of individuals of the member population having N number of chronic conditions or more as identified through the condition groupings. For example, it may be desirable, when analyzing for cost impact related to chronic conditions, to have at least X portion of the member population with no more than three chronic conditions. In some embodiments, the threshold is based at least in part on statistical impact to the analysis. For example, in relation to costs, groupings corresponding to less than a threshold portion of the total allowed costs of the control group may be discarded.

Turning to FIG. 9C, in some implementations, a sub-method 930 begins with calculating, for each condition identifier, at least one corresponding impact value (932). In some embodiments, calculating the impact value includes calculating costs associated with each of the groupings (e.g., within the control group and/or the member group). For example, claims data 146 of FIG. 1 may be accessed to correlate cost to each identifier within a certain grouping. In further embodiments, correlating each grouping with an impact includes calculating mortality rates and/or adverse reaction rates associated with each of the groupings (e.g., within the control group and/or the member group). For example, the reactions/mortality data 144 of FIG. 1 may be accessed to correlate reaction and/or mortality to each member corresponding to the identifier(s) within a certain grouping. Calculating the impact value, in some embodiments, includes assessing clinical observations based on survey data and/or response data automatically collected through physiological monitoring of a patient. For example, in clinical trials, employee assistance program (EAP) services, and/or other mental health services, surveys and clinical observations may be collected to assess patient response to various treatments. In another example, in virtual clinical trials or in treating patients with a wearable medical device, physiological sensor data may be collected to assess a patient's response to a treatment. The impact value, for example, may represent average or median values across multiple members with the same condition. In some embodiments, outlier values within the multiple members may be discarded when calculating the impact value(s).

If multiple impact values were calculated for each condition identifier of at least a portion of the condition identifiers (934), in some implementations, the multiple impact values are combined into a single value (936). The single value may represent a weighting of types of impact values such that, for example, cost is balanced against mortality rate and/or adverse reaction rate. In an illustrative example, cost may be adjusted by an actuarial quantity calculated for each of a set of adverse reaction severities. In other embodiments, rather than combining the values into a single value, the multiple values may be combined into a vector format for later calculations.

In some implementations, the condition identifiers are arranged by impact to the underlying analysis (940). In the example of a single value of cost, the costs per person for each of the condition identifiers may be arranged in numeric order. In some implementations, if multiple impact values were calculated, arranging the condition identifiers impact may include scaling the impact (e.g., by number of adverse reaction categories) to produce a distribution of impact values across the condition identifiers.

In some implementations, based on the arranging, the most impactful conditions are selected (942). The groupings, for example, may be ranked and/or rated based upon impact to the analysis. The most impactful conditions, further, may be analyzed for similarity (e.g., the second and seventh most impactful conditions are each sub-groups of a particular grouping, so grouping those two sub-groupings for analysis may be beneficial). In some embodiments, a threshold number is selected. The threshold number, in some examples, may be based in part on a size of the member population, a size of the control population, a type of analysis desired, a user specified threshold, and/or a default threshold.

In some implementations, the member population is matched to the control population based on the selected condition identifiers (944). The matching, for example, may be performed as described in relation to the method 200 of FIG. 2.

If the selected condition identifiers result in fewer than desired matches for at least one condition (946), in some implementations, one or more condition identifiers are removed (948). To improve quality of matching, the set of top condition identifiers may be adjusted, through iterating (944 through 948) on differing combinations of condition identifiers (e.g., by removing one or more condition identifiers at each iteration).

In some implementations, once the member population has been matched to the control population in a manner deemed suitable for analysis (946), the sub-method 930 completes and the matches are either stored or provided to a requesting program.

Returning to FIG. 9A, if the number of condition identifiers does not exceed a threshold (922) (e.g., all condition identifiers can be used for matching in the construct of the present analysis), in some implementations, the member population is matched to the control population based on the condition identifiers (950). The matching may be performed as described in relation to operations 944 through 948 of FIG. 9C.

The method 900 is illustrated as a particular set of operations. In other embodiments, more or fewer operations may be included in the method 900. For example, in some embodiments where multiple formats of identifiers are gathered in relation to the member population, the method 900 may include mapping a portion of the condition identifiers and/or prescription identifiers into a single format for analysis. In another example, the method 900 may include organizing sub-groupings based in part on weighting factors. In illustration, in analysis focusing on a cancer study, codes related to that type of cancer may be refined, while other co-morbidity codings may be analyzed at a more general degree of specificity.

Although described in relation to a particular series of operations, in other embodiments, one or more steps of the method 900 may be performed in a different order and/or in parallel. For example, analysis of different types of identifier codings may be performed in parallel. In another example, organizing sub-groupings as part of general groupings (909) may be performed after determining that the number of condition identifiers exceeds a threshold (922) such that more generalized groupings may be identified on an as-needed basis. Other modifications of the method 900 and its sub-methods 910 and 930 are possible while remaining in the spirit and scope of the process.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory (i.e., non-volatile) computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2, the process 400 of FIG. 4, the method 900 of FIG. 9A, the sub-method 910 of FIG. 9B, and/or the sub-method 930 of FIG. 9C.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the clients 104, patient research data sources 105, claims data sources 106, medical record sources 108, and/or benefits coverage sources 110 and the health care efficiency analysis platform 102 of FIG. 1, the data sources 412, 414, 416, 418, 420, 422, 424 and the corresponding engines 402, 404, and 406 of FIG. 4, and/or the report generation engine 410 and the computing system of the display 442 of FIG. 4.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display may enable presentation of the screen shots illustrated, in some examples, in FIG. 5A through FIG. 5C, FIG. 8A through FIG. 8C, and/or the tables illustrated in FIG. 6 and/or FIG. 7.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google™ Cloud Storage or Amazon™ Elastic File System (EFS™), may store processed and unprocessed data supplied by systems described herein. For example, the contents of the data repository 112 of FIG. 1 may be maintained in a database structure.

The systems described herein may communicate with the cloud computing environment through a secure gateway. In some implementations, the secure gateway includes a database querying interface, such as the Google BigQuery™ platform or Amazon RDS™. The data querying interface, for example, may support access by the engines of the health care efficiency analysis platform 102 and the data repository 112.

While certain embodiments have been described, these embodiments have been presented by way of example only, and are not intended to limit the scope of the present disclosures. Indeed, the novel methods, apparatuses and systems described herein can be embodied in a variety of other forms: furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A system for automatically building a control population closely matched to a member population and applying the control population in establishing medical outcome comparison metrics with the member population, the system comprising:
a non-volatile computer readable storage medium storing an association between each of a plurality of demographic group indicators and a plurality of medical condition group indicators and one or more individuals within a control population comprising a plurality of individuals, wherein
each indicator of the plurality of demographic group indicators corresponds to a respective demographic category of a plurality of demographic categories, each demographic category comprising two or more demographic groups, and
each indicator of the plurality of medical condition group indicators corresponds to a respective medical condition category of a plurality of medical condition categories, each medical condition category comprising two or more groups; and
processing circuitry configured to perform a plurality of operations, the operations comprising,
for each given member of a plurality of members of a member population, accessing respective demographic information of the given member,
using the respective demographic information of the given member, classifying the given member into a respective demographic group of each given demographic category of at least a portion of the plurality of demographic categories according to the respective two or more demographic groups of the given respective demographic category, wherein
the plurality of demographic categories in which the given member is classified comprise at least one of age, gender, geography, one or more socio-economic factors, and/or one or more environmental exposure factors, and
classifying comprises storing, to a member record of the given member, a set of demographic group indicators comprising a respective demographic group indicator of each demographic category of the at least the portion of the plurality of demographic categories,
accessing respective medical data of the given member,
using the respective medical data of each given member, classifying the given member into at least one respective medical condition group of each given medical condition category of at least a portion of the plurality of medical condition categories according to a set of medical condition groups of the given respective medical condition category, wherein
classifying comprises storing, to the member record of the given member, a set of medical condition group indicators comprising a respective medical condition group indicator of each medical condition category of the at least the portion of the plurality of medical condition categories, and
using the set of demographic group indicators and the set of medical condition group indicators stored to the member record of the given member, comparing the given member to at least a portion of the plurality of individuals of the control population to identify whether one or more closest matching individuals exist in the control population, wherein
the one or more closest matching individuals each have a corresponding set of demographic group indicators and a corresponding set of medical condition group indicators corresponding to at least a portion of the set of demographic group indicators and at least a portion of the set of medical condition group indicators of the respective member, and
when the comparing results in the identifying of the one or more closest matching individuals,
the given member is included in an analysis member population comprising a subset of the member population, and
at least one of the one or more matching individuals is included in a benchmark population, and
providing the analysis member population, the benchmark population, and at least one of a) costs data, b) medical outcomes data, or c) insurance claims data associated with each member of the analysis member population and each member of the benchmark population for comparison analysis.

2. The system of claim 1, wherein the plurality of medical condition categories comprises at least one of a chronic condition category, a habits category, a vitals category, or a laboratory results category.

3. The system of claim 2, wherein the habits category comprises participation in one or more employer-sponsored health programs.

4. The system of claim 2, wherein the chronic condition category is a total number of chronic conditions category including a no chronic conditions group, a one chronic condition group, and at least one group representing multiple chronic conditions.

5. The system of claim 1, wherein:
the non-volatile computer readable storage medium stores a plurality of demographic records corresponding to each individual of the plurality of individuals of the control population; and
the operations further comprise
receiving at least one customized demographic category comprising at least two customized groups, and
using the respective demographic information of each respective individual of the plurality of individuals of the control population, classifying the respective individual into a respective demographic group of the at least two customized groups of each category of the at least one customized demographic category.

6. The system of claim 1, wherein:
at least one demographic category of the portion of the plurality of demographic categories comprises a hierarchical sub-categorization structure, wherein
the hierarchical sub-categorization structure comprises progressively finer levels of sub-categorization; and
identifying the one or more closest matching individuals of the control population comprises, for at least one member of the plurality of members of the member population, iteratively
seeking to identify at least one matching individual of the control population at a given level of the hierarchical sub-categorization structure for the at least one demographic category, and
upon failing to identify at least one matching individual of the control population, selecting a coarser demographic subcategory level of the at least one of the plurality of demographic categories.

7. The system of claim 1, wherein the plurality of demographic categories comprises a geography category having sub-categorizations corresponding to larger and larger geographic areas.

8. The system of claim 1, wherein:
the member population is composed of participants in a medical study corresponding to a particular medical condition; and
the operations further comprise analyzing the medical outcomes data of the analysis member population in view of the medical outcomes data of the benchmark population to derive statistical differences in one or more medical outcomes.

9. The system of claim 1, wherein:
the member population is composed of participants in an employer-sponsored health program; and
the operations further comprise analyzing the insurance claims data of the analysis member population in view of the insurance claims data of the benchmark population to derive statistical differences in healthcare utilization.

10. The system of claim 1, wherein identifying the one or more closest matching individuals of the control population comprises using a subset of the plurality of medical condition group indicators identified as having a greatest impact to the comparison analysis.

11. The system of claim 10, wherein the operations further comprise analyzing at least one of the costs data, the medical outcomes data, and the insurance claims data to identify, among the member population, a portion of the plurality of medical condition group indicators having the greatest impact to the comparison analysis.

12. The system of claim 11, wherein identifying the portion of the plurality of medical condition group indicators having the greatest impact to the comparison analysis comprises analyzing, for each respective demographic group of at least one demographic category of the plurality of demographic categories, the at least one of the costs data, the medical outcomes data, and the insurance claims data to identify, among a subset of the member population belonging the respective demographic group, a respective portion of the plurality of medical condition group indicators having the greatest impact to the comparison analysis for the respective demographic group.

13. The system of claim 12, wherein the plurality of demographic categories comprises at least one of age or gender.

14. The system of claim 12, wherein the operations further comprise selecting, from the respective portion of the plurality of medical condition group indicators for each demographic group of each respective demographic category of the portion of the plurality of demographic categories, a final set of most impactful medical condition group indicators affecting members of the member population belonging to all groups of the respective demographic category.

15. The system of claim 14, wherein the portion of the plurality of medical condition group indicators having the greatest impact to the comparison analysis are selected to represent at least 90% of a variation in outcome between the member population and the control population.

16. The system of claim 12, wherein the respective portion of the plurality of medical condition group indicators comprises at least fifteen medical condition indicators.

17. The system of claim 11, wherein the plurality of medical condition group indicators comprises a total number of indicators of at least one thousand medical condition group indicators.

18. The system of claim 11, wherein identifying the portion of the plurality of medical condition group indicators having the greatest impact comprises reducing a total number of indicators by at least a factor of 10.

19. The system of claim 1, wherein the operations comprise applying at least one machine learning model trained to categorize medical conditions into groupings to separate coded medical data by layers of specificity in a medical coding structure.

20. The system of claim 1, wherein identifying the one or more closest matching individuals of the control population comprises applying at least one machine learning model trained to perform dimensionality reduction of the plurality of demographic group indicators and the plurality of medical condition group indicators to identify a most relevant subset of members of the member population.
